(19) United States
(12) Patent Application Publication
(10) Pub. No.: US 2024/0173489 A1
(43) Pub. Date: May 30, 2024

(54) Portable ultrasonic nebulizer

(71) Applicant: Synergy Life Science, Inc., Buford, GA (US)

(72) Inventors: Chris Gerlach, Buford, GA (US); Mauricio Amaya, Buford, GA (US); Zachary Hudson, Buford, GA (US); Adam Gerlach, Buford, GA (US); Aaron Hudson, Buford, GA (US); Tim Harrington, St. Petersburg, FL (US)

(21) Appl. No.: 18/060,063

(22) Filed: Nov. 30, 2022

(51) Int. Cl. A61M 11/00

(52) U.S. Cl. CPC A61M 11/005; A61M 2205/121; A61M 2205/587

(57) ABSTRACT

The portable ultrasonic nebulizer is a device that uses a high-frequency piezoelectric oscillator to create an aerosolized medication for inhalation. The medication is typically a liquid, but can also be a suspension, emulsion, or solution. The portable ultrasonic nebulizer is formed from two primary components: a controller and a cartridge. The controller includes the electronics and power source for the operation of the device. The cartridge includes the reservoir in which the medication is held, as well as the associated components for delivery of the medication, including the wick and piezoelectric oscillator. The cartridge is single-use, or disposable. Thus, the user keeps the controller, to which she joins cartridges as desired, whether to change medications, or as a refill.

### FIELD

This invention relates to the field of devices for the delivery of medication by inhalation and more particularly to a portable nebulizing device.

### BACKGROUND

The delivery of medication via inhalation is an important and common means of administering medication, in particular for patients with asthma or other respiratory conditions. Medication is delivered directly to the lungs, providing ready access to the blood and therefore to the body.

Devices for delivery of medication via inhalation include vaporizers and nebulizers.

An ultrasonic nebulizer is a type of nebulizer that uses ultrasonic waves to create a fine mist of medication, the mist subsequently inhaled. The mist is created by a piezoelectric crystal vibrating at a high frequency, causing the liquid medication to be aerosolized into tiny droplets.

The high frequency vibrations of the ultrasonic nebulizer create a small, consistent particle size that is ideal for inhalation. The particle size is small enough to reach the small airways and alveoli in the lungs, where the medication can be most effective.

The drawbacks of existing ultrasonic nebulizers include their lack of affordability and their bulky size.

Electronic cigarettes, or e-cigarettes, are also used to deliver medications or chemicals to the lungs. In an e-cigarette, a heating device vaporizes a liquid solution, creating an aerosolized collection of droplets for inhalation by the user. But the process of heating the liquid solution can damage it, changing its chemical properties and reducing its effectiveness.

What is needed is a portable ultrasonic vaporizer that can effectively deliver medication to a user while providing the convenience of an electronic cigarette.

### SUMMARY

The portable ultrasonic nebulizer is a device that uses a high-frequency piezoelectric oscillator to create an aerosolized medication for inhalation.

The medication is typically a liquid, but can also be a suspension, emulsion, or solution.

In the preferred embodiment, the medication is carried to the piezoelectric oscillator by an absorbent wick. The wick is in contact with a reservoir of medication, the medication drawn from the reservoir, into the wick, and finally into contact with the piezoelectric element.

In alternative embodiments, in place of a wick, alternative means are used to carry medication to the piezoelectric oscillator.

The portable ultrasonic nebulizer has multiple advantages over other types of nebulizers. Specifically, its portability makes it convenient for use anywhere, and the fine mist makes it effective for delivery of medication.

The preferred liquid for carrying the medication is water, without oil. As an alternative to pure water, a saline solution may also be used. No heat is used during the aerosolization, avoiding chemical changes to the liquid or its associated medication. The quantity of medication delivered to the user can be measured, allowing for metered doses.

The portable ultrasonic nebulizer is formed from two primary components: a controller and a cartridge. The controller includes the electronics and power source for the operation of the device. The cartridge includes the reservoir in which the medication is held, as well as the associated components for delivery of the medication, including the wick and piezoelectric oscillator.

The cartridge is single-use, or disposable. Thus, the user keeps the controller, to which she joins cartridges as desired to change medications, or when a cartridge is empty.

The piezoelectric oscillator is integrated into the replaceable cartridge. By replacing the oscillator with every cartridge change the user is provided with a clean oscillator. This ensures contamination is not carried from one medication to another.

In a first embodiment, the piezoelectric oscillator is a round-ring atomizer plate with multiple apertures through which atomized liquid is discharged. For example, a multiplicity of five micrometer holes through which medication from the wick is drawn, atomized, and discharged to the mouthpiece of the device.

The replaceable cartridge and the controller—also known as the reusable controller—communicate in order to manage the delivery of medication. The replaceable cartridge includes a code or other means of indicating the type of medication present in the reservoir. The code can include other information, such as the appropriate quantity to constitute a dose. This medication and dosing information is communicated to the controller, which in turn determines how to best operate the piezoelectric oscillator to dispense the appropriate quantity of medication.

To assist a user with the delivery of metered doses, an indicator light is visible during operation of the unit. In the preferred embodiment, the indicator light takes the form of a ring visible around the circumference of the device. The controller monitors medication delivery, makes a determination based on the appropriate quantity of medication that constitutes a single dose, then triggers illumination of an LED when a metered dose has been dispensed. The light of the LED is carried through a light pipe to a ring, where the light is visible to the user.

In an alternative embodiment, the controller communicates wirelessly with an app to provide reporting of various metrics associated with use. For example, the device may include sensors that track air flow rate, vapor particle size, air quality, cartridge contents, battery performance, and related device performance values. These values are communicated wirelessly by, for example, Bluetooth, to a user's mobile device. The mobile device tracks and stores this data, optionally providing the data to a central server. The server processes this data to provide the user with: useful metrics regarding performance; to remind the user when refills are required; and to allow future versions of the device to incorporate improvements based on real-life use data.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates an assembled isometric view of the portable ultrasonic nebulizer with cartridge.

FIG. 2 illustrates an assembled view in cross section of the portable ultrasonic nebulizer with cartridge.

FIG. 3 illustrates an assembled view in cross section focusing on the cartridge of the portable ultrasonic nebulizer with cartridge.

FIG. 4 illustrates a view without the housing of the portable ultrasonic nebulizer with cartridge.

FIG. 5 illustrates a partially disassembled view of the controller and cartridge of the portable ultrasonic nebulizer with cartridge.

FIG. 6 illustrates a second partially disassembled view of the controller and cartridge of the portable ultrasonic nebulizer with cartridge.

FIG. 7 illustrates an exploded view of the cartridge of the portable ultrasonic nebulizer with cartridge.

FIG. 8 illustrates a view of the piezoelectric oscillator and wick assembly of the portable ultrasonic nebulizer with cartridge.

FIG. 9 illustrates a view showing how the cartridge fits into the controller and housing of the portable ultrasonic nebulizer with cartridge.

FIG. 10 illustrates a second view showing how the cartridge fits into the controller and housing of the portable ultrasonic nebulizer with cartridge.

FIG. 11 illustrates a third view showing how the cartridge fits into the controller and housing of the portable ultrasonic nebulizer with cartridge.

FIG. 12 illustrates a view of the charging port of the portable ultrasonic nebulizer with cartridge.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

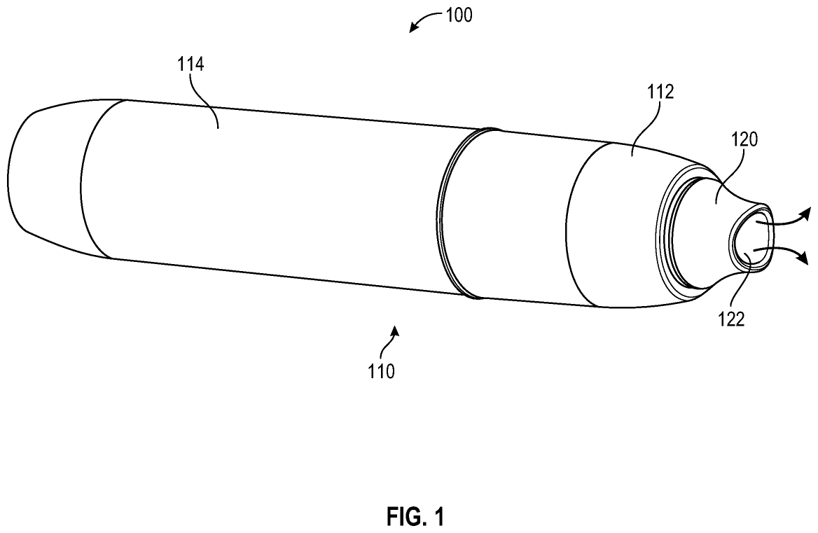

Referring to FIG. 1, an assembled isometric view of the portable ultrasonic nebulizer with cartridge is shown.

The portable ultrasonic nebulizer with cartridge 100 includes a housing 110 formed from a front section 112 and a rear section 114.

The user can place her lips against mouthpiece 120 to inhale through the vapor channel 122.

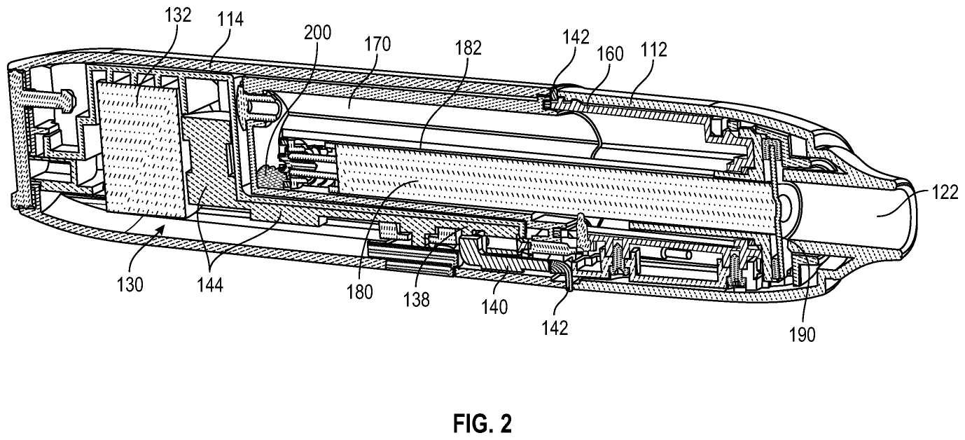

Referring to FIG. 2, an assembled view in cross section of the portable ultrasonic nebulizer with cartridge is shown.

The cartridge 16o includes an internal reservoir 170. The fluid 200 is in contact with the wick 180, the wick 180 carrying the fluid 200 to the piezoelectric oscillator 190. The fluid 200 is then atomized, exiting through the vapor channel 122.

The controller 130 includes battery 132 and control board 144. When a dose has been delivered, control board 144 causes illumination of light source 138, the light passing through light pipe 140 to light ring 142.

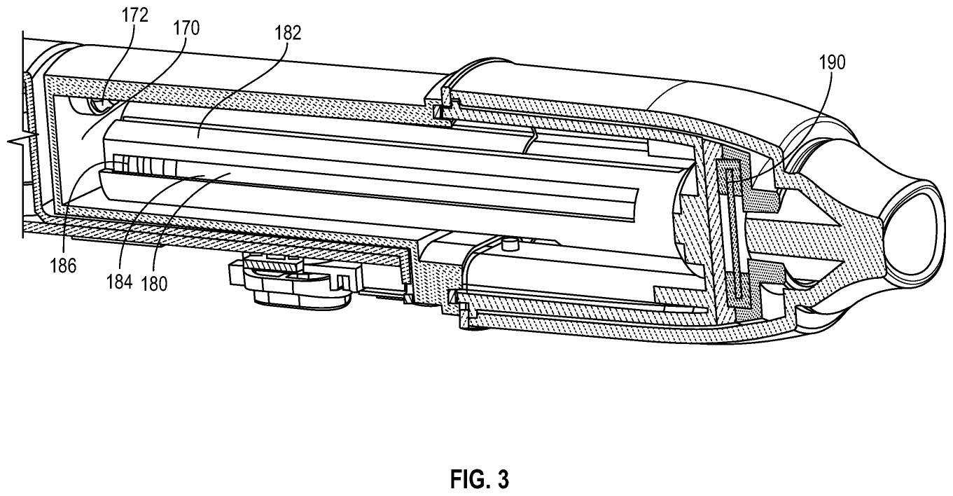

Referring to FIG. 3, an assembled view in cross-section focusing on the cartridge of the portable ultrasonic nebulizer with cartridge is shown.

The reservoir 170 is plugged using an optional reservoir plug 172. Fluid 200 (see FIG. 2) within the reservoir 170 contacts the wick 180, carrying fluid to the piezoelectric oscillator 190. Compression of the wick 180 against the piezoelectric oscillator 190 is maintained by spring 186 to ensure consistent contact between the piezoelectric oscillator 190 and the wick 180, therefore ensuring consistent delivery of medication. The position of a wick 180 is maintained by wick sleeve 182, with separations creating channels 184, to allow the fluid 200 to contact the wick 180.

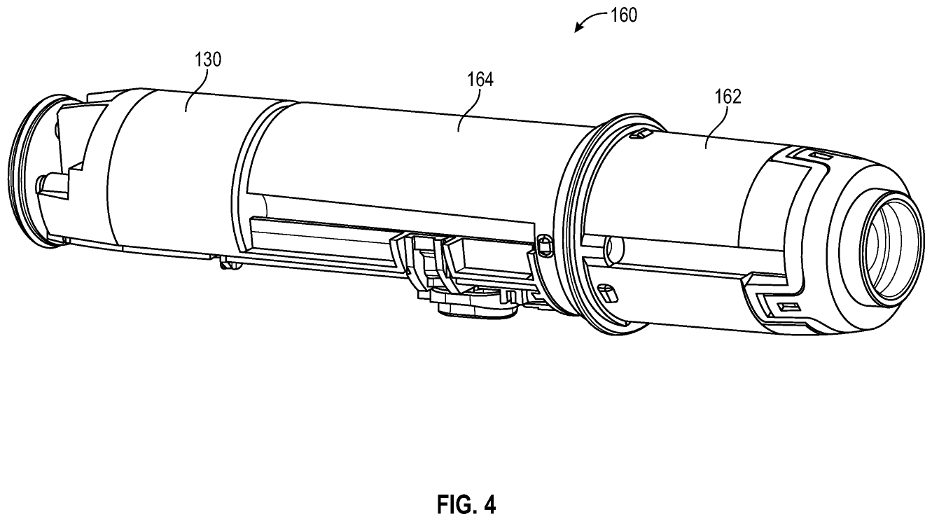

Referring to FIG. 4, a view without the housing of the portable ultrasonic nebulizer with cartridge is shown.

Cartridge 160 is formed from a first-half 162 and a second-half 164. The cartridge 160 interfaces with controller 130 to create an operational portable ultrasonic nebulizer with cartridge 100.

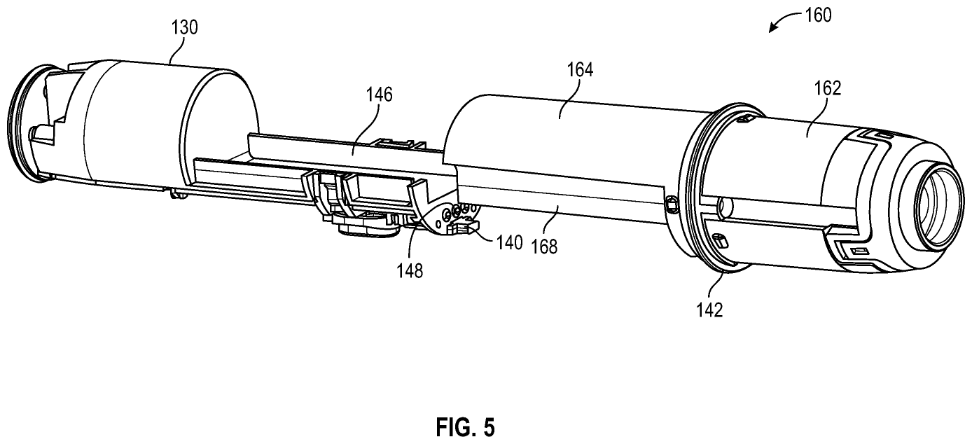

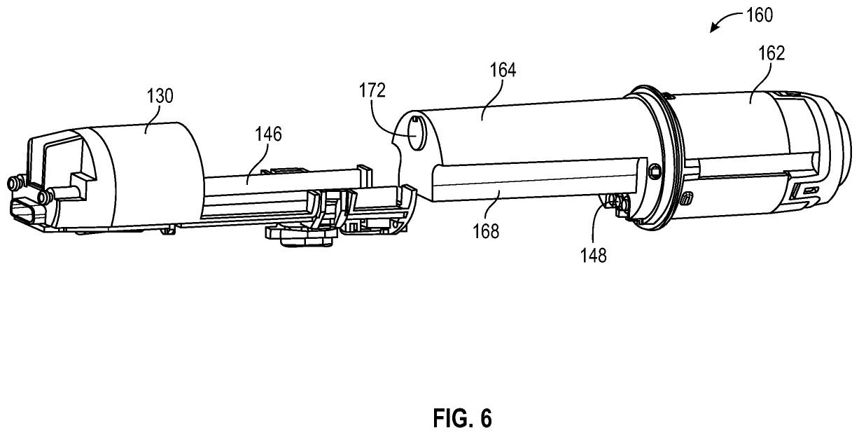

Referring to FIGS. 5 and 6, two partially disassembled views of the controller and cartridge of the portable ultrasonic nebulizer with cartridge are shown.

To align the controller 130 and cartridge 160, protrusion 168 of the cartridge 160 slides into channel 146 of the controller 130.

Light pipe 140 is visible protruding from the controller 130, the light pipe carrying light to the light ring 142 of the cartridge 160.

Also visible are electrical contacts 148, carrying electrical power and signal data from the controller 130 to the cartridge 160, specifically for the piezoelectric oscillator 190 (see FIG. 2).

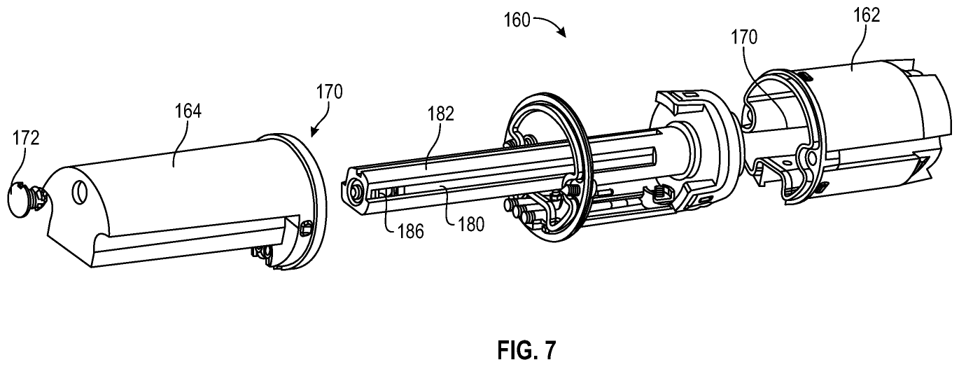

Referring to FIG. 7, an exploded view of the cartridge of the portable ultrasonic nebulizer with cartridge is shown.

The reservoir 170 is shown formed from first half 162 and second-half 164 of cartridge 160. The optional reservoir plug 172 can be removed for filling or emptying reservoir 170. A wick 180 is held within wick sleeve 182, with pressure maintained by spring 186.

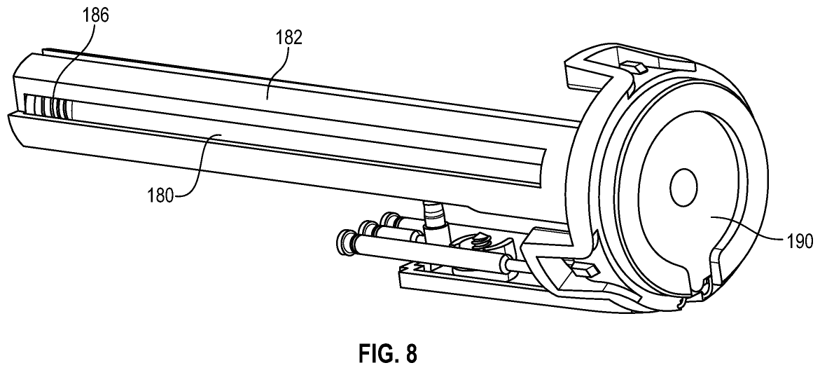

Referring to FIG. 8, a view of the piezo electric oscillator and wick assembly of the portable ultrasonic nebulizer with cartridge is shown.

The wick 180 is held within a wick sleeve 182, with pressure maintained against piezoelectric oscillator 190 by spring 186.

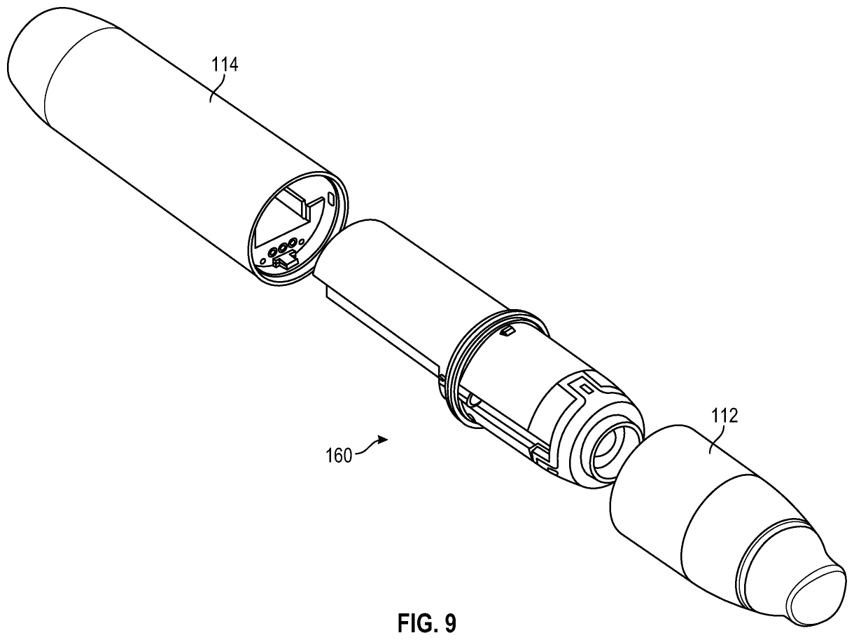

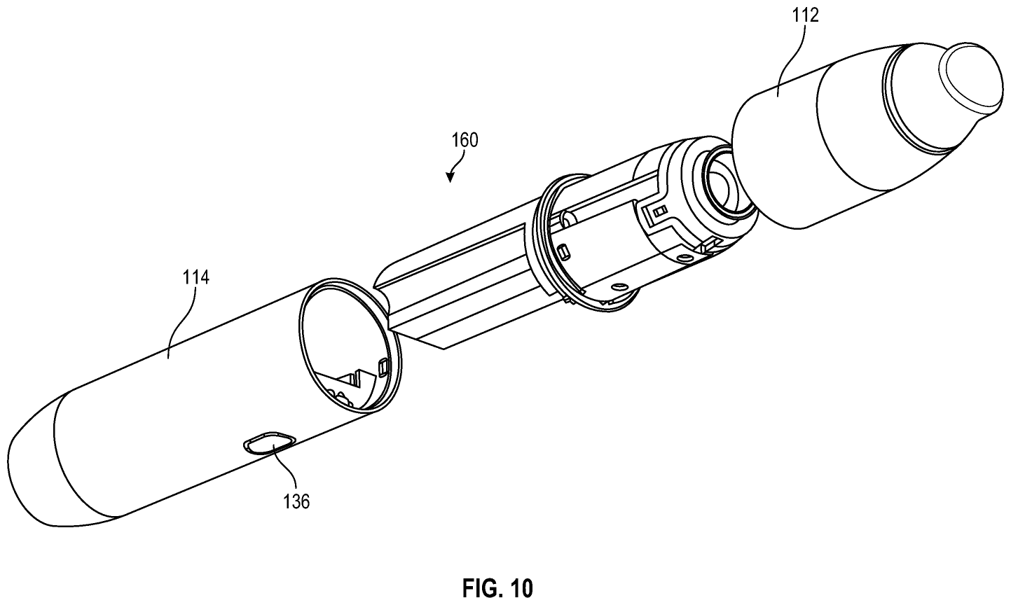

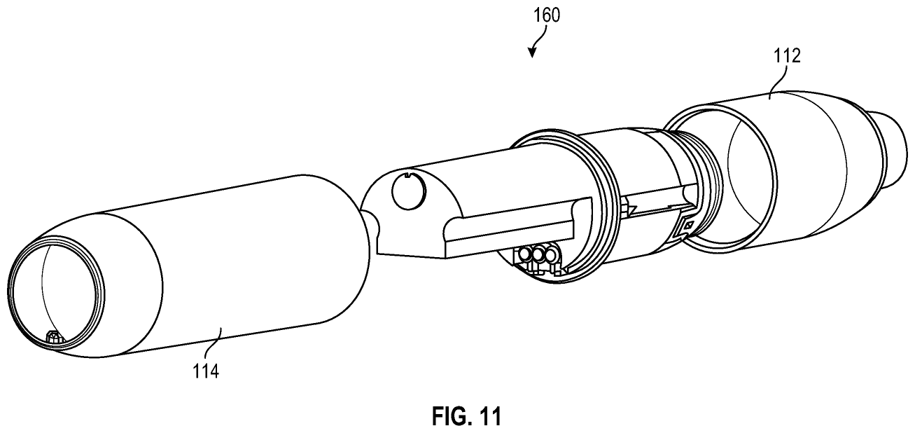

Referring to FIGS. 9, 10, and 11, three views showing the cartridge fitting into the controller and housing of the portable ultrasonic nebulizer with cartridge are shown.

The front section 112 and rear section 114 of the housing 11o (see FIG. 1) separate to grant access to the cartridge 160. Power button 136 is actuated by the user to operate the portable ultrasonic nebulizer with cartridge 100.

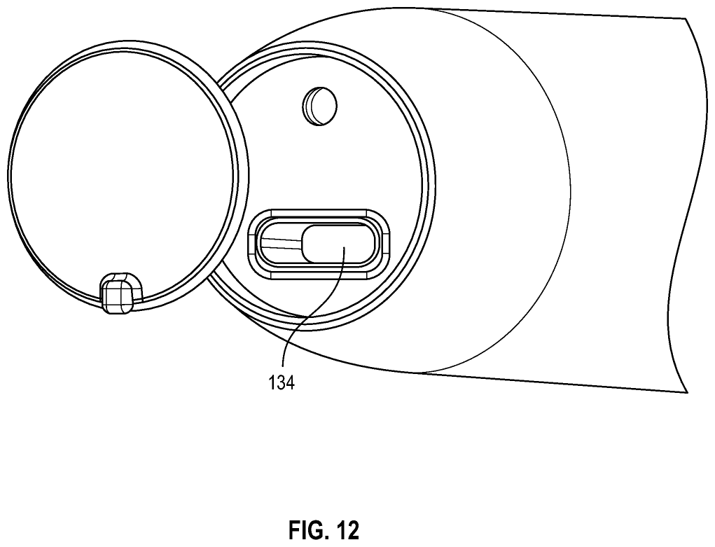

Referring to FIG. 12, a view of the charging port of the portable ultrasonic nebulizer with cartridge is shown.

The charging connector 134, preferably a USB C port, is accessible for the user to charge the device.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A portable nebulizing device that integrates medication delivery with a reservoir to avoid cross-contamination, the portable nebulizing device comprising:
a replaceable cartridge that connects to a reusable controller;
the replaceable cartridge including a reservoir, the reservoir prefilled with medication;
the replaceable cartridge including a piezoelectric oscillator, the piezoelectric oscillator acting to nebulize the medication, forming a mist for inhalation;
the reusable controller including a battery and associated electronics for operation of the piezoelectric oscillator;
whereby a user selects a replaceable cartridge, connects it to the reusable controller, and uses the reusable controller to operate the piezoelectric oscillator, causing the piezoelectric oscillator to dispense medication as a mist.

2. The portable nebulizing device of claim 1, the replaceable cartridge further comprising:
a wick;
the wick placed within the reservoir;
the wick carrying medication from the reservoir to the piezoelectric oscillator, where the medication is nebulized;
whereby the wick ensures a consistent flow of medication to the piezoelectric oscillator.

3. The portable nebulizing device of claim 2, wherein:
the wick is supported by a wick sleeve;
the wick sleeve including channels that allow liquid from within the reservoir to be pass through the wick to the piezoelectric oscillator.

4. The portable nebulizing device of claim 3, further comprising:
a spring;
the spring compressing the wick within the wick sleeve;
the spring maintaining pressure of the wick against the piezoelectric oscillator;
whereby the spring ensures consistent contact between the wick and the piezoelectric oscillator to ensure consistent delivery of medication.

5. The portable nebulizing device of claim 1, further comprising:
a light source:
light source having one or more states, including an on state where light is emitted, and an off state where light is not emitted;
the light source operated by the reusable controller;
the reusable controller changing between the one or more states of the light source to indicate the piezoelectric oscillator has dispensed one dose of the medication held within the reservoir
wherein the replaceable cartridge communicates to the reusable controller a type of medication held within the reservoir;
whereby the reusable controller can operate the light source to indicate when an appropriate dosage of medication has been dispensed.

6. The portable nebulizing device of claim 1, further comprising:
a fluid within the reservoir;
the fluid being water-based;
a medication within the fluid;
the medication having a set concentration;
whereby dispensing a certain volume of fluid results in dispensation of a known quantity of medication.

7. A portable nebulizer for administering medication, the portable nebulizer comprising:
a replaceable cartridge;
a reservoir within the replaceable cartridge;
a piezoelectric oscillator within the replaceable cartridge;
the piezoelectric oscillator causing nebulization of liquid medication from within the reservoir, resulting in a mist that is discharged;
the piezoelectric oscillator integrated into the replaceable cartridge, such that when the replaceable cartridge is replaced, the piezoelectric oscillator is also replaced;
whereby replacing the piezoelectric oscillator when the replaceable cartridge is switched avoids cross-contamination of different liquid medications.

8. The portable nebulizer for administering medication of claim 7, the replaceable cartridge further comprising:
a wick;
the wick placed within the reservoir;
the wick carrying medication from the reservoir to the piezoelectric oscillator, where the medication is nebulized;
whereby the wick ensures a consistent flow of medication to the piezoelectric oscillator.

9. The portable nebulizer for administering medication of claim 8, wherein:
the wick is supported by a wick sleeve;
the wick sleeve including channels that allow liquid from within the reservoir to be drawn into the wick to the piezoelectric oscillator.

10. The portable nebulizer for administering medication of claim 9, further comprising:
a spring;
the spring compressing the wick within the wick sleeve;
the spring maintaining pressure of the wick against the piezoelectric oscillator;
whereby the spring ensures consistent contact between the wick and the piezoelectric oscillator to ensure consistent delivery of medication.

11. The portable nebulizer for administering medication of claim 7, further comprising:
a reusable controller;
the reusable controller connecting to the replaceable cartridge to create an operable portable nebulizer;
a light source:
light source having one or more states, including an on state where light is emitted, and an off state where light is not emitted;
the light source operated by the reusable controller;
the reusable controller changing between the one or more states of the light source to indicate the piezoelectric oscillator has dispensed one dose of the medication held within the reservoir
wherein the replaceable cartridge communicates to the reusable controller a type of medication held within the reservoir;
whereby the reusable controller can operate the light source to indicate when an appropriate dosage of medication has been dispensed.

12. The portable nebulizer for administering medication of claim 7, further comprising:
a fluid within the reservoir;
the fluid being water-based;
a medication within the fluid;
the medication having a set concentration;
whereby dispensing a certain volume of fluid results in dispensation of a known quantity of medication.

13. A portable device for administering medication via nebulization, the portable device including:
a controller section;
a battery within the controller section;
a cartridge;
the cartridge affixed to the controller section;
a reservoir within the cartridge;
a piezoelectric oscillator permanently affixed to the cartridge, whereby the piezoelectric oscillator is disposed of with the cartridge and controller when the reservoir has been emptied of medication;
whereby the controller and cartridge are replaced simultaneously, avoiding cross-contamination and thus ensuring accurate dosing.

14. The portable device for administering medication via nebulization, of claim 13, the cartridge further comprising:
a wick;
the wick placed within the reservoir;
the wick carrying medication from the reservoir to the piezoelectric oscillator, where the medication is nebulized;
whereby the wick ensures a consistent flow of medication to the piezoelectric oscillator.

15. The portable device for administering medication via nebulization, of claim 14, wherein:
the wick is supported by a wick sleeve;
the wick sleeve including channels that allow liquid from within the reservoir to be drawn into the wick to the piezoelectric oscillator.

16. The portable device for administering medication via nebulization, of claim 15, further comprising:
a spring;
the spring compressing the wick within the wick sleeve;
the spring maintaining pressure of the wick against the piezoelectric oscillator;
whereby the spring ensures consistent contact between the wick and the piezoelectric oscillator to ensure consistent delivery of medication.

17. The portable device for administering medication via nebulization, of claim 13, further comprising:
a light source:
light source having one or more states, including an on state where light is emitted, and an off state where light is not emitted;
the light source operated by the controller section;
the controller section changing between the one or more states of the light source to indicate the piezoelectric oscillator has dispensed one dose of the medication held within the reservoir
wherein the cartridge communicates to the controller section a type of medication held within the reservoir;
whereby the controller section can operate the light source to indicate when an appropriate dosage of medication has been dispensed.

18. The portable device for administering medication via nebulization, of claim 13, further comprising:
a fluid within the reservoir;
the fluid being water-based;
a medication within the fluid;
the medication having a set concentration;
whereby dispensing a certain volume of fluid results in dispensation of a known quantity of medication.
